(19) United States
(12) Patent Application Publication
(10) Pub. No.: US 2006/0177850 A1
(43) Pub. Date: Aug. 10, 2006

(54) Particle-based multiplex assay system with three or more assay reporters

(76) Inventors: Mack Schermer, Belmont, MA (US); Mark N. Bobrow, Lexington, MA (US); Wayne F. Patton, Newton, MA (US)

Correspondence Address: CESARI AND MCKENNA, LLP, 88 BLACK FALCON AVENUE, BOSTON, MA, 02210, US

(21) Appl. No.: 11/321,374

(22) Filed: Dec. 29, 2005

Related U.S. Application Data
(60) Provisional application No. 60/640,882, filed on Dec. 30, 2004.

(51) Int. Cl. C12Q 1/68; C12M 1/34; G01N 33/551

(52) U.S. Cl. 435/6; 436/524; 435/287.2

(57) ABSTRACT

A system and method for developing and utilizing particle-based n-multiplexed assays that include three or more reporters utilizes n particle sets that are associated with particle identification images or labels (IDs) that differ between sets. The encoded particles for a given set are coated with a specific binding member, or in the case of the sandwich assay with coupled capture and detector binding pair members, to form particle types. The sets of particle types are then pooled, and aliquots of the particle types are removed to assay vessels. Next, samples with three or four reporter molecules are supplied to the respective vessels. After one or more incubation periods, the particles are supplied to a reader system, which determines the particle IDs to identify the particle types and also detects the reporter signals. The reader system includes multiple excitation lasers that excite the various reporters in sequence or in parallel, to supply associated signals to one or more detectors. Emission filters and wavelength discriminators are included such that a given detector receives at a given time the signals associated with a single assay binding label. The system further develops greater capacity sandwich assays by assigning subsets of capture and detector antibody pairings to the three or four reporters, respectively. The system performs greater numbers of differential RNA expressions based on the use of the three or more reporters, with one or more reporters assigned to the reference sample and the other reporters assigned to respective test samples. The system and method are also capable of performing greater numbers of SNPs utilizing primer extension reactions, by assigning different color reporters to the respective nucleotides or terminators.

### CROSS-REFERENCE TO RELATED APPLICATION

The present application claims the benefit of U.S. Provisional Patent Application Ser. No. 60/640,882, which was filed on Dec. 30, 2004, by Mack Schermer, Mark N. Bobrow and Wayne F. Patton for a PARTICLE-BASED MULTIPLEX ASSAY SYSTEM WITH THREE OR MORE ASSAY REPORTERS and is hereby incorporated by reference.

### BACKGROUND OF THE INVENTION

1. Field of the Invention

The invention relates generally to multiplex assay systems and, more particularly, to particle-based multiplex assay systems with a plurality of reporters.

2. Background Information

The simultaneous measurement of concentrations or amounts of multiple analytes in a complex biological sample has many applications in the fields of drug discovery, medical research and biological research. A common multiplex assay format is the planar microarray, on which a plurality of members of specific binding pairs, such as antibodies or nucleic acid sequences, are immobilized in various configurations on a planar support. Typically, a printing or lithographic process is used to deliver the binding entities as a plurality of spots, and the positions of the respective spots correspond to the identities of the entities. Common types of microarrays include RNA expression arrays and antibody arrays.

A given array is arranged on a planar support made of glass or silicon, or on a thin gel or membrane layer coated on top of the support. Typically, complex biological samples, which contain unknown mixtures of analytes that are complementary to the specific immobilized binding pair members, are applied to the array and allowed to incubate. The immobilized binding pair members capture or mate with at least a fraction of the complementary specific binding pair members in the samples.

A labeling mechanism is also utilized, such that the mated binding pairs produce detectable signals, usually optical signals such as fluorescence or a change in color. The signals are thereafter analyzed to determine the relative concentrations of the analytes. The labeling can be accomplished by many methods. The simplest ones utilize chemical or enzymatic labeling of all or most of the potential binding molecules in the sample. More specific results may be obtained using a more complicated assay development. One example is a “sandwich” assay that utilizes a second binding pair member that differs from those previously immobilized on the array but which has a specific affinity to the captured substances at each array location. The second binding pair member and an associated label are added into the assay in a known manner, as a separate step in the assay development operations.

The multiplexed assays may also be used for detecting single-base mutations, also known as “SNPs” (single nucleotide polymorphisms), in complex nucleic acid samples. SNPs may be detected on microarrays, for example, by hybridization discrimination between immobilized probes with match and single-base-mismatch sequences complementary to the target sequences in the sample, as described in U.S. Pat. No. 6,300,063 to Lipshutz et al., which is incorporated herein by reference. A more robust SNP assay is performed by single-base extension as described in U.S. Pat. No. 5,888,818 to Goelet and U.S. Pat. No. 6,013,431 to Soderland et al., which are incorporated herein by reference. The single-based extension assay has been performed in multiplex on microarrays using a 4-color primer extension assay. See Hirschorn et al., “SBE-TAGS: An array-based method for efficient single-nucleotide polymorphism genotyping”, PNAS 97-22, Oct. 24, 2000, p. 12164-12169, which is incorporated herein by reference.

Multiplexed assays for simultaneous measurements of a plurality of proteins are also known. See, e.g., Haab et al., “Protein microarrays for highly parallel detection and quantization of specific proteins and antibodies in complex solutions,” Genome Biology 2001 2(2): research 0004.1-0004.13, which is incorporated herein by reference. To produce the simultaneous measurements, two samples are labeled with two different fluorescent dyes, the labeled samples are mixed, and the mixture is assayed in two-color multiplex on the microarray. The two-color difference, or differential assays, whether for nucleic acids, proteins or other analytes, produces ratiometric measurements, which have certain advantages. In particular, the ratios cancel out some sources of assay errors, such as, for example, differences in labeling efficiency or binding assay affinity for the different analytes.

While microarrays are extremely useful, they must be precisely produced in order that the results can be correctly interpreted. The creation of the arrays is complex and requires specialized, and costly, equipment as well as particular skills in the people who operate the equipment. Further, the collection of data from the microarrays similarly requires skilled operations and/or complex, specialized equipment, particularly given the necessary tolerances associated with the location and sizing of the spots.

The arrays must be printed with well-controlled nanoliter or picoliter amounts of immobilized capture molecules at each spot, such that the signal-producing potential of each spot is constrained. This presents a challenge, in part, because the concentrations of the solutions being printed change with, for example, evaporation, during the printing process. Further, local hydrophobicity variations of the array substrate, on a micro scale, can have relatively large effects on the size of the printed spots, which also affects the concentrations of the binding pair members.

To overcome certain of the challenges in producing the microarrays, in-situ synthesis of the microarray elements may be performed using optical lithography, to selectively deprotect defined regions in an array in order to build up polymers of, for example, a few dozen elements. See, Fodor et al., “Light-directed, spatially addressable parallel chemical synthesis”, Science, Feb. 15, 1991, p. 767-773. While the in-situ synthesis produces high-quality microarray elements, or spots, the process requires the use of even more complex and costly equipment, as well as time consuming setup operations.

The collection of data from an incubated microarray requires the imaging of the array, that is, the measuring of an appropriate optical property (e.g. fluorescence, color, etc.) across the array on a pixel-by-pixel basis to form an electronic image. The image must then be segmented into “spot” and “background” areas, and the values of the pixels in the spot segments must to be consolidated into respective values representing the intensities of the spots.

The imaging process itself is quite challenging, since the exact size, shape and location of each spot may vary within substantial tolerances that are associated with the limitations of the array printing processes, as discussed briefly above. The spot printing process, for example, generally produces spots that have rather substantial variations in concentrations when examined on the scale of typical microarray imaging pixels, i.e., from about 3 μm to about 30 μm. Further, individual pixels may span a spot and a boundary area, and thus, be associated with differing surrounding intensity levels. When producing a single value for a given spot, the imaging process must appropriately compensate for the variations across the spot, and also detect and discard the boundary spanning pixels.

For these and other reasons microarrays are associated with certain reproducibility problems and their use has thus been largely excluded to date from diagnostics and other critical settings. Further, the expense and complexities associated with producing the microarrays discourages their use for low-volume situations.

There are a variety of known non-microarray multiplexed assay systems that are useful when greater reproducibility is required. The systems of interest use encoded beads or particles (both of which are referred to herein collectively as “particles”), to identify the respective binding pair members that are used to capture analytes from the samples. A given binding pair member is immobilized on identically encoded particles in a solution phase, such that the binding pair member can be identified based on the identification of the associated encoded particle, that is, by the encoded “particle ID.” Multiplex assays are then constructed by pooling several sets of the coated and encoded particles, which also referred to herein as respectively “particle types.”

Thereafter, portions of the pooled multiplexed assay, with approximately equal numbers of the respective particle types, are transferred to assay vessels, such as microplate wells, vials, or tubes. Samples that include respective labeling reagents are then added to the assay vessels. After sufficient incubation, the binding assay signal is read optically by an instrument that can also optically determine the encoded identifications of the respective particle types.

The way in which the binding assay signals are read depends in part on how the particles are encoded. The particles may be encoded based on size and/or color, by marks etched on the particles, or by images produced within the particles, such as holograms.

In one system of particular interest, the particles are encoded by impregnating them with mixtures of various proportions of two or more fluorescent dyes. The assay binding signal is then reported by a fluorescent label that has an excitation and emission wavelength that is substantially separated from those associated with the particle-identification dyes. The assay binding signal and the particle ID signal associated with the respective particles are read by a flow-cytometer type of instrument, which draws the particles from the assay vessel and individually interrogates them. The instrument, which optically interrogates the particle as the particle passes through a reading capillary, first obtains the particle ID and then determines the intensity of the single fluorescence assay binding signal. This system has been implemented commercially by the Luminex Corp. (Austin, Tex.) as its xMAP product line. The product line is currently used for a variety of assay types in research, drug discovery and in some FDA-approved diagnostic applications.

Another known system of particular interest utilizes glass particles that are encoded with holographic barcodes. The barcodes are read optically by providing an excitation beam at a wavelength, polarization and incident angle that corresponds to the encoding of the particles. The barcodes have an advantage of readily encoding a relatively large number of particle types, as well as an advantage of the environmental robustness.

While these systems overcome problems associated with reproducibility, by eliminating the tolerances associated with the spot printing processes of the microarray systems, there are distinct limitations associated with the known particle-based systems. For instance, the known particle-based systems operate with particle ID optical signals and one or two labeling fluorescence signals. Such systems are thus particularly well suited for a variety of nucleic acid hybridization assays and immunoassays, but not well suited for differential protein expression assays, differential gene expression, SNPs utilizing primer extensions, and so forth, which require additional independently measurable labeling signals. Therefore, what is,needed are methods and systems for performing a variety of multiplexed particle-based assays utilizing three or more labels.

### SUMMARY OF THE INVENTION

The current invention is a system and method for developing and utilizing particle-based n-multiplexed assays that include three or more reporters. The system and method provide a user with enhanced capabilities of performing various differential expression multiplexed assays simultaneously or, in the case of an improved sandwich assay development, performing assays with enhanced specificity that include greater numbers of multiplexed encoded and coated particle sets and/or subsets.

The system and method utilizes n particle sets that are associated with particle IDs that differ between sets. The encoded particles for a given set are coated with a specific binding member, or in the case of the sandwich assay with coupled capture and detector binding pair members, to form particle types. The sets of particle types are then pooled, and aliquots of the particle types are removed to assay vessels. Next, samples with three or four reporter molecules are supplied to the respective vessels.

After one or more incubation periods, the particles are supplied to a reader system, which determines the particle IDs to identify the particle types and also detects the reporter signals. The reader system includes multiple excitation light sources, such as laser or other devices with controlled wavelengths and optical power, such as LEDs, SLDs, broadband sources with excitation filters, and so forth. The light sources excite the various reporters in sequence or in parallel, to supply associated signals to one or more detectors. Emission filters and wavelength discriminators are included such that a given detector receives at a given time the signals associated with a single assay binding label.

The system further develops greater capacity sandwich assays by assigning subsets of capture and detector antibody pairings to the three or more reporters, respectively, as discussed in more detail below.

The system and method are capable of simultaneously performing greater numbers of differential RNA expressions than known prior systems based on the use of the three or more reporters, with one or more reporters assigned to the reference sample and the other reporters assigned to respective test samples. The system and method are also capable of performing greater numbers of SNPs utilizing primer extension reactions, by assigning different color reporters to the respective nucleotides or terminators.

### BRIEF DESCRIPTION OF THE DRAWINGS

The invention description below refers to the accompanying drawings, of which:

FIG. 1 is a flow diagram of the operations of preparing and obtaining results from a plurality of particle-based multiplexed assays three or more reporters;

FIG. 2 is a schematic view of a holographically encoded multiplex assay particle;

FIG. 3 is a schematic view of the particle of FIG. 3 with molecules of a specific protein binding pair member, such as an antibody, immobilized thereon to produce a corresponding particle type;

FIG. 4 is a schematic view of the particle of FIG. 3 with a plurality of immobilized specific protein binding molecules captured by their complementary proteins;

FIG. 5 is a schematic view of the particle of FIG. 4 with a plurality of captured proteins labeled by a specific binding of a secondary binding pair member;

FIGS. 6A and 6B are schematic diagrams of a reading system that interrogates the particle of FIG. 3 or FIG. 4 sequentially with multiple excitation beams;

FIG. 7 is a schematic diagram of a filter wheel for use in the systems of FIGS. 6A and 6B;

FIG. 8 is a schematic diagram of a reading instrument that interrogates the particle simultaneously with multiple excitation beams;

FIG. 9 is a schematic diagram of an alternative reading instrument that interrogates the particle simultaneously with multiple excitation beams.

### DETAILED DESCRIPTION OF AN ILLUSTRATIVE EMBODIMENT

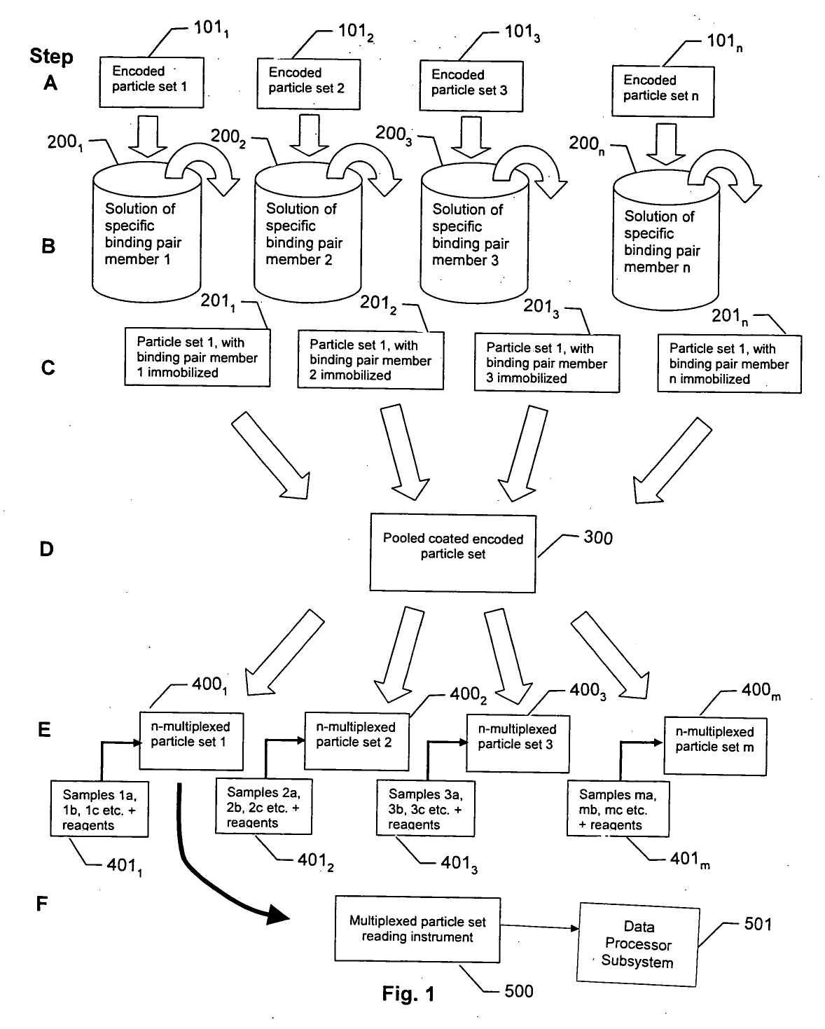

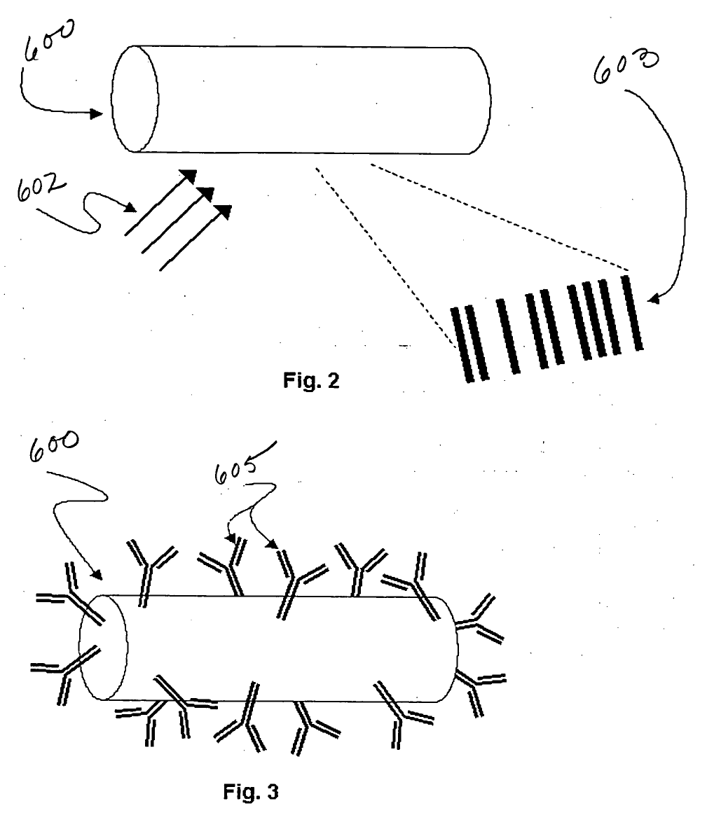

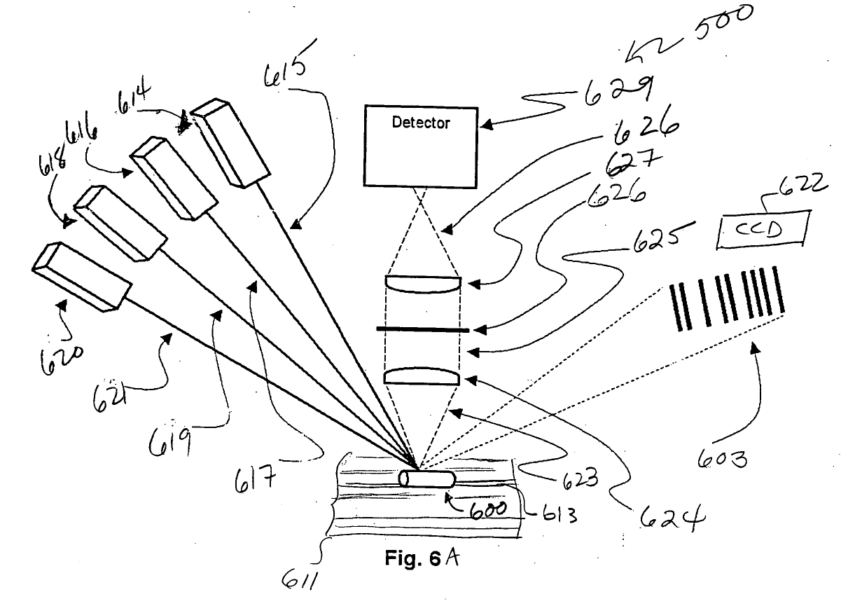

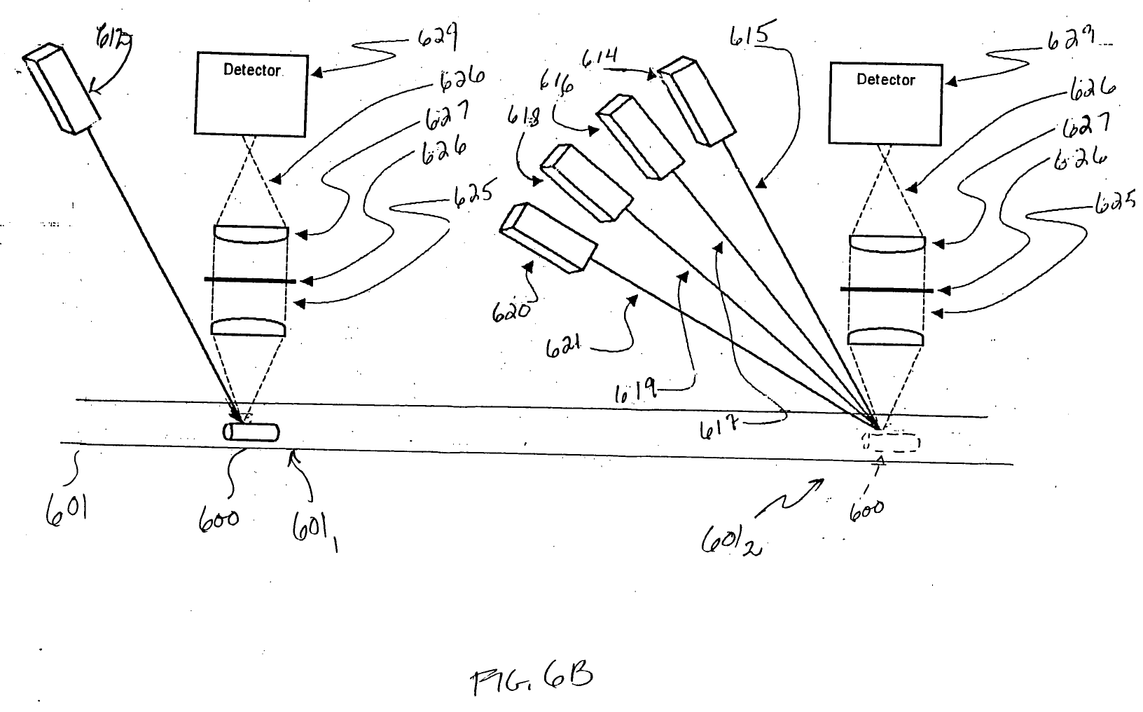

Referring to FIG. 1, the steps of preparing and performing an encoded particle multiplex assay involving n analytes of interest and three or more reporter molecules are described. The various steps are thereafter described in more detail with reference to FIGS. 2-5, and reading systems for the multiplexed assays are described with reference to FIGS. 6A-9.

Referring now to FIG. 1, in step A particles are encoded in a known manner to produce n particle sets 101₁ . . . 101ₙ, in which the particles are encoded with particle IDs. The particles in a given set are identically encoded with a particle ID, and no two sets utilize the same particle ID. The particle IDs may be, for example, holographic bar codes, etched gratings, ratios of two colors of fluorescent dyes, quantum dot emissions, and so forth.

In Step B the respective encoded particle sets 101₁ . . . 101ₙ are reacted with solutions 200₁ . . . 200ₙ of specific binding pair members, for example, protein-specific antibodies or oligonucleotides, which are complementary to particular analytes of interest. This produces particle sets 201₁ . . . 201ₙ in which the respective specific binding pair members are immobilized on the encoded particles. Each set thus includes an associated encoded and coated particle or “particle type,” and no two sets include the same particle types.

As discussed in more detail below with reference to FIG. 5, “sandwich” assays may be utilized to provide more specificity to the assay. Accordingly, second specific binding pair members, or detector antibodies, may be supplied to certain or all of the encoded particle sets in an additional step in the assay development.

In Step C the particle types in a given set 201₁ . . . 201ₙ are removed from the immobilizing solution by, for example, pipetting, filtering, centrifugation, or any other standard laboratory process for separating solid particles from liquids. In step D, the particle types from the respective sets 201₁ . . . 201ₙ are mixed together, typically in an aqueous buffer, to produce a pooled particle set 300.

In Step E, aliquots of the particle types are taken from the pooled particle set 300 and supplied to respective assay vessels (not shown), to produce a plurality of n-multiplexed particle sets 400₁ . . . 400ₘ. The assay vessels may be, for example, microplate wells, tubes, vials, and so forth.

The aliquots will have nominally identical numbers of particles and nominally equal populations of each of the n particle types. The distributions may deviate from the nominal conditions due, for example, to randomness and tolerances associated with either or both of the mixing of the particle types within the pooled particle set and the aliquotting process itself. The aliquots are sized such that they contain, for each particle type, a selected multiple of a minimum number required to generate a valid assay signal. Typically, the number is selected from ranges of 5-20 or 500 to 5000 replicates of each of the particle types, depending on how the imaging operations are performed. For example, the range of 500 to 5000 replicates is used for operations that read the respective particles once, e.g., operations involving two-color encoded particles. The range of 5-20 replicates is used for holographically encoded particles that are each read in more than one location. The holographic particles thus generate ten data points per particle, or 50-200 data points over the 5-20 replicate range.

Also in step E, various samples and associated reagents, referenced collectively by the numerals 401₁ . . . 401ₘ, are supplied to the corresponding assay vessels. The samples to be assayed, and the associated reagents, such as labeling reagents containing three or four reporting molecules, washing reagents, and so forth, are supplied to the respective assay vessels in the proper sequence, and the solution contained in the assay vessel is then allowed to incubate for one or more incubation periods, as appropriate.

In step F, the n-multiplex particle set, which now has the labeled assayed analytes bound to each particle, is removed from the assay vessel and transferred to a reading instrument 500. The reading instrument, which is described in more detail with reference to FIGS. 6A-9 below, optically interrogates each particle, to read therefrom the particle ID and the associated assay signals. The assay signals from replicate particles types within a given n-multiplexed particle set may be consolidated in the reading instrument, to produce the signals that are provided to a data processing computer 501 for analysis. The data processing computer then analyses the signals in a known manner. Alternatively, the assay signals from each particle may be supplied to the computer, which then performs the consolidation before performing the analysis. As discussed, three or four reporter, or label, molecules are utilized in the n-multiplexed assays, such that a given multiplex assay is capable of separately measuring a plurality of the analytes of interest in the samples supplied to the assay vessels.

The particles may be encoded in a number of ways to associate them with particle IDs that can be optically discerned by the reading system 500. The reading instrument must also read the three or more reporter signals from the respective particles either serially or in parallel, to produce associated signals for later analysis. Before discussing the reading system in more detail, we describe encoded particles that are particularly well suited for use with the system.

Referring now to FIG. 2, an encoded particle 600 consists essentially of a section of an optical fiber that has a barcode or other identifier recorded therein, in the form of, for example, a hologram or a diffraction grating. To reproduce the particle ID, here a holographic bar code image 603, the particle 600 is interrogated by a beam of parallel light 602 that is provided at a controlled wavelength, polarization and incidence angle.

The particle 600 is shown as cylindrical, however, the particle may have other shapes. The particle may, for example, be made from a length of glass fiber that has a diameter between about 10 μm and 100 μm and a length between about 25 μm and 250 μm. The hologram is preferably recorded in the glass such that the holographic image diverges as the image projects from the particle, to produce at a distance of about 10 to about 100 mm away from the particle an image 603 that is several millimeters long. As discussed in more detail below, the projected image, here the barcode, can then be read by a low-resolution imaging array such as a charge-couple device (CCD) camera 622 (FIG. 6A).

Referring now to FIG. 3, the encoded particle 600 is coated (steps B and C FIG. 1), such that antibodies 605 attach to or immobilize on a particle surface 604. In an embodiment that involves assaying proteins, for example, the antibodies are specific to particular binding sites on specific proteins.

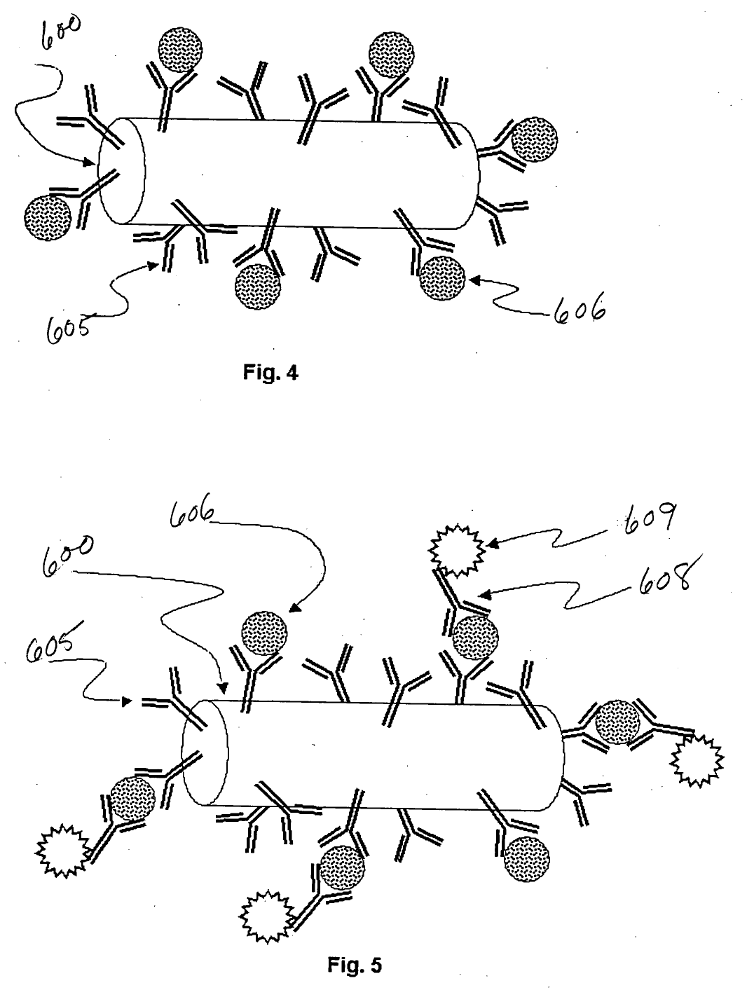

Referring now to FIG. 4, the coated particle 600 is depicted after assay incubation with a sample (step E of FIG. 1), in which certain of the immobilized antibodies 605 are bound to their complementary proteins 606. As discussed, the proteins are labeled with a detectable molecule such as a fluorophore, chromophore, fluorescent protein, quantum dot or the like, in order to provide a signal to the reading instrument 500. Alternatively, such labeling may be applied chemically or enzymatically to the proteins, nucleic acid or other specific binding pair members in the samples. The labeling may be applied prior to or after the inclusion of the sample in the multiplexed assay.

To enhance the specificity of the assay, the assay may be developed as a sandwich assay, in which a label or reporter molecule conjugates to a secondary, or detector, antibody. Referring now to FIG. 5, the detector antibodies 608, which are labeled with reporter molecules 609, specifically bind to proteins 606 that have been captured by the immobilized primary, or capture, antibodies 605. The protein is thus sandwiched between the capture and the detector antibodies. The detector antibodies are labeled and the capture antibodies are not. Thus, the reading system detects those antibodies that are bound to the captured proteins, rather than those that are bound to the antibody coating on the particle.

As discussed above, the reporter signals associated with the particles from the n-multiplexed assays are read by a reading instrument 500 (step F, FIG. 1). As shown in FIGS. 6A-B, a sequential reading instrument includes four excitation lasers 614, 616, 618, and 620 that direct four excitation beams 615, 617, 619, and 621 to the multiplex assay particle 600. For ease of understanding, the drawing depicts the system optics and omits the associated mechanical and electrical subsystems that operate in known manners to, for example, move and focus the optics, move the particles, record the signals, shutter the lasers, and so forth.

In the example depicted in drawing, the four excitation beams are directed to substantially the same location on the particle 600, which is held in a selected alignment relative to the beams by a groove 613 on a particle reading plate 611. One of the beams has the wavelength, polarization and incidence angle needed to reconstruct the holographic barcode image 603 that identifies the particle type then being read. As discussed, the holographic image is preferably constructed such that the image can be projected in a manner that allows the barcode to be read by a low-resolution instrument, such as the CCD camera 622.

In addition, the reading instrument 500 reads the assay binding signals produced by the fluorescent assay reporter molecules that are excited by the excitation beams 615, 617, 619, 621. The reporter molecules, which may be, for example, fluorophores, fluorescent proteins or quantum dots, act as point sources and emit fluorescence light in all directions from their locations on the surface of the particle. The reading instrument, through an objective lens 624, captures the emissions that are directed toward the lens. The captured emissions are represented in the drawing by dotted lines 623.

The objective lens 624 collimates the captured emissions into a parallel emission beam 625, which is, in turn, directed to a wavelength discrimination component 626. The wavelength discrimination component, which may be, for example, a filter, selectively passes to a detector 629 a detection beam 626 that consists essentially of the signals from one of the fluorescent reporters. The detector, which operates in a known manner, produces an electrical signal that corresponds to the intensity of the passed reporter signal. A second, or detector, lens 627 may also be included to reduce the diameter of the detection beam, to match the diameter to the dimensions of an input window (not shown) of the detector or, as appropriate, to optionally accommodate a confocal pinhole (not shown). For each laser, conventional shutters (not shown) may be included to supply the excitation beams sequentially to the particle. At the same time, the system operates in a known manner to switch into the optical path to the detector a wavelength discriminator component that has the appropriate peak emission and pass band wavelengths. Alternatively, a geometric beamsplitter (not shown) may be used in place of the wavelength discriminator component. A geometric beam splitter of particular interest is described in U.S. Pat. No. 6,441,379, which is incorporated herein by reference. To simplify the system a single wavelength discriminator component, such as a filter that passes all of the wavelengths of interest, may be used. However, the trade off is a reduction in the signal detection efficiency.

The excitation lasers 614, 616, 618, 620 may be of many types including, for example, helium-neon, argon, diode, diode-pumped solid state, and frequency doubled solid state. The associated delivered laser power is typically in the range of 1 to 50 mW.

The objective lens 624 preferably has a numerical aperture in the range of about NA 0.2 to NA 0.75. While objective lenses with higher numerical apertures collect more fluorescence light, and thus, improve the associated signal-to-noise ratio, the lenses have smaller depth of focus and other geometric constraints that complicate the opto-mechanical design of the system. There is thus a trade-off between the complexity of the reader and the selection of the lens numerical aperture.

The detector 629 is selected for good signal to noise performance at low light levels. The detector may be, for example, a photomultiplier tube, an avalanche photodiode, and so forth.

The four-laser detection system depicted in FIG. 6 may be simplified if the system is designed for use with quantum dots as reporter molecules rather than conventional fluorophores. Quantum dots have broad excitation spectra and narrow emission spectra. Typically, three, four or more different quantum dot types with distinguishable emission spectra can be excited by a single relatively short wavelength. Usually, a single excitation wavelength between about 350 nm and 488 nm is used and emission peaks can be produced in the range of 500 nm to 750 nm. Thus, different wavelength discriminator components can be switched into the optical path to the detector to sequentially detect the three or four reporters that are excited by a single excitation beam.

In FIG. 6B, the particle 600, which in this example is encoded using two dyes, flows along a path 601 and is interrogated first at a location 601₁ in order to detect the particle ID. As shown, the same objective lens, wavelength discriminator component and so forth, may be used at this location. However, the system optics may be optimized for the detection of the two ID colors of interest and, for example, an emission filter may be used in place of the wavelength discriminator component.

At a next location 601₂, the particle is interrogated by each of the lasers 614, 616, 618 (if three reporter molecules are used) and also 620 (if four molecules are used), to read the assay binding labels.

The systems of FIG. 6A and 6B may be constructed with three excitation lasers or with more than four excitation lasers, such that the system can read, in addition to the particle ID, three or more than four assay reporter signals, respectively.

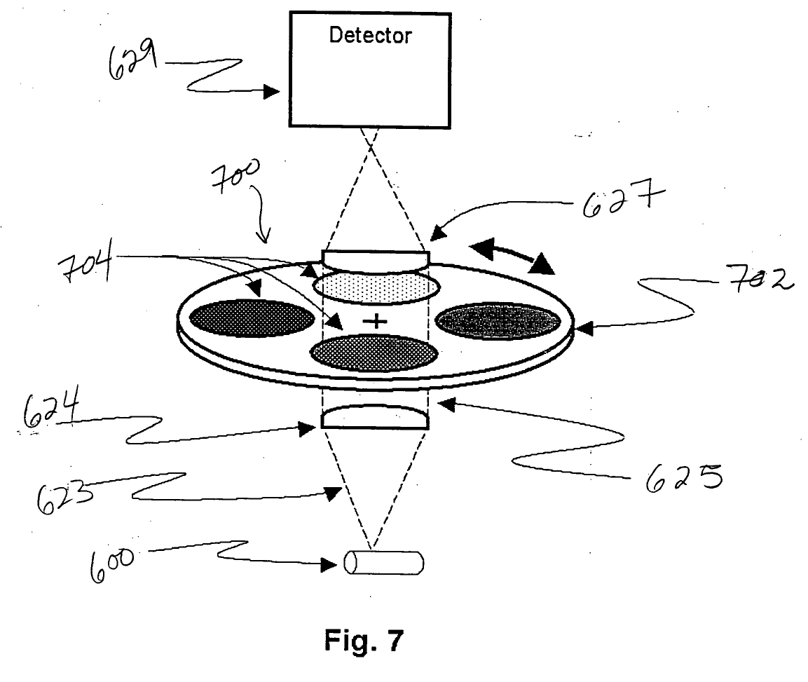

Rather than switching different wavelength discrimination components 626 into the optical path, the component may be replaced with a variable wavelength discrimination component, such as a filter wheel 700 depicted in FIG. 7. The filter wheel consists essentially of a rotatable holder 702 that is configured to hold a desired number of emission filters 704. In the example, the holder is configured to hold four filters. The filters are bandpass filters, and a given filter has a transmission peak that approximately coincides with the peak emission wavelength of one of the fluorescent reporter molecules and a passband width that is small compared to the differences between the peak emission wavelengths of the reporter molecules. The filter wheel of the example rotates to four positions, to allow the detector to read four labels associated with the particle 600. For ease of understanding, the mechanical structure, which operates in a known manner to rotate the filter wheel, is not shown in the drawing.

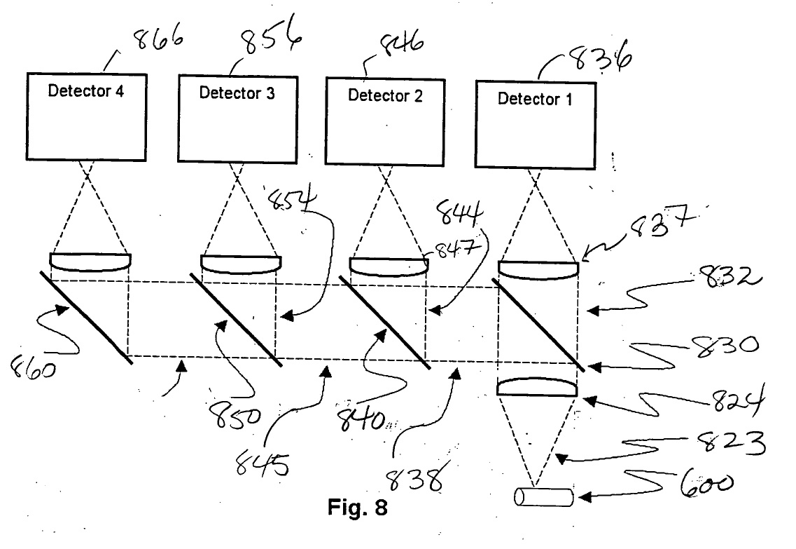

Referring now to FIG. 8, an alternative example of the four-color detection optics of the reading system 500 is depicted. The four excitation lasers 614, 616, 618, 620 and associated excitation beams 615, 617, 619, 621 are omitted from the drawing for ease of understanding. An objective lens 824 collects a cone of emission light 823 and collimates it into a parallel mixed emission beam 825. In the example, the reporters, or labels, have wavelengths that vary from a longest for a first label to shortest, shorter and short wavelengths for the second, third and fourth labels, respectively.

A first dichroic 830 transmits to a first detector 834 as a first detection beam 832 light with wavelengths close to the wavelength of the first label and also light with longer wavelengths. The first detector 836 operates in a known manner to produce an electrical signal that corresponds to the intensity of the first detection beam. An optional detector lens 837 may be included in the optical path of the first detection beam, to reduce the diameter of the beam to match the dimensions of an input window (not shown) of the detector. Further, an emission filter (not shown) may be included in the optical path, to pass the wavelength of interest, and thus, block the longer wavelengths from reaching the detector.

The first dichroic 830 also reflects shorter wavelength light, as a reflected emission beam 838, to a second dichroic 840. The second dichroic, in turn, reflects to a second detector 846, as a second detection beam 844, light in the wavelengths close to the wavelength of the second label. The second detector 846 operates in a known manner to produce an electrical signal that corresponds to the intensity of the second detection beam 844. An optional detector lens 847 may also be included.

The second dichroic 840 also transmits light that has longer wavelengths as a transmitted emission beam 845 to a third dichroic 850. The third dichroic reflects to a third detector 856, as a third detection beam 854, light with wavelengths close to the third label, and the third detector operates in a known manner to produce an electrical signal that corresponds to the intensity of the third detection beam. The third dichroic also transmits light with longer wavelengths to a mirror 860, which reflects the transmitted light to a fourth detector 866. The mirror is included such that the geometries of the detector locations are essentially the same. However, the light transmitted by the third dichroic may instead be transmitted to the fourth detector. The fourth detector operates in a conventional manner to produce an electrical signal that corresponds to the intensity of the light reflected or transmitted to the detector.

As described, the embodiment of FIG. 8 has four detectors 836, 846, 856, and 866 and operates with four reporters. The system may instead include three detectors and operate with three reporters, or more than four detectors and operate with additional reporters. Further, the dichroics 830, 840, 850 and 860 are the only wavelength discrimination components shown in the drawing. However, emission filters may be used between the respective dichroics and detectors, to reduce crosstalk. While this architecture allows simultaneous measurement of four reporter wavelengths using a single, shared objective lens, there is a trade-off of the corresponding economy with the optical inefficiencies and added complexities associated with the use of the dichroic elements.

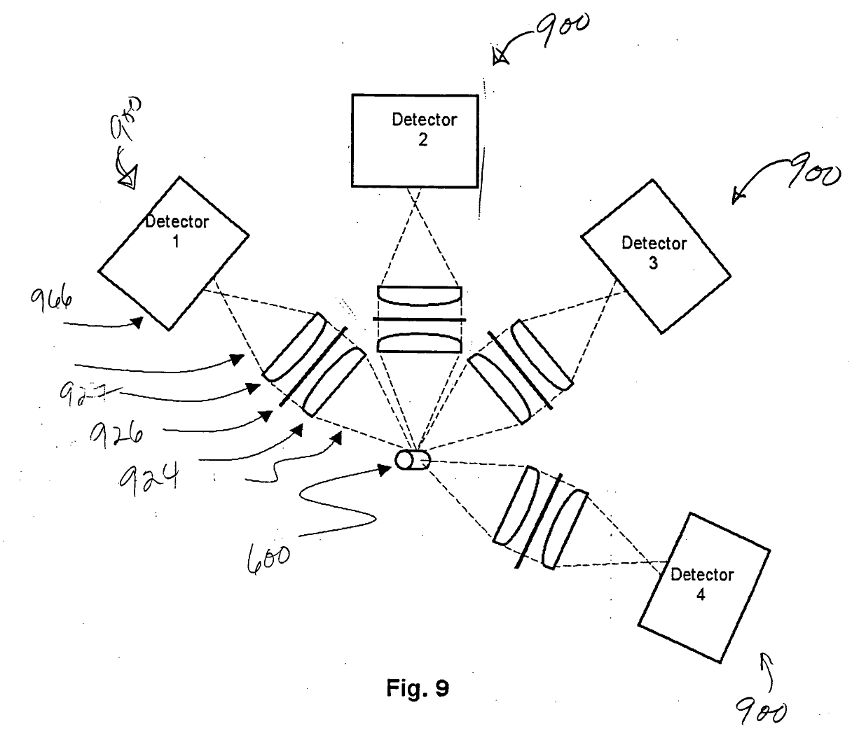

FIG. 9 depicts another arrangement of the reading system optics, in which four detection modules 900 are utilized. Each detection module includes an objective lens 924, a fixed emission filter 926, a detector lens 927 and a detector 966. The detection modules are substantially identical except for the transmission wavelengths of the respective emission filters. The fixed emission filter in a given detector module is selected to pass, or transmit, the signals for a single label or reporter molecule. Thus, the architecture allows simultaneous detection of four reporters and eliminates the optical inefficiencies of the dichroic approach. However, the trade-off is the use of multiple objective lenses, which adds to the cost of the system.

The system is depicted in FIG. 9 with four detection modules 900. However, the system may include three or more that four detection modules, to simultaneously detect three or more than four labels.

The current system provides methods and systems for performing particle-based multiplexed assays and independently measure three or more labels or reporter molecules in addition to the particle IDs. Described below are specific examples in which the current system provides a user with new capabilities to perform in a multiplexed particle-based assay what could previously be performed using microarrays. Further, we provide a new method for working with differential RNA expression assays and a new method for developing and working with multiplexed sandwich assays.

A. Differential Protein Expression Assays

The use of three labels in a particle-based multiplexed assay can provide significantly greater utility in differential protein expression assays than the use of two labels. A reference sample (typically a normal sample) is labeled with a first label, a test sample (typically a diseased or treated sample) is labeled with a second label, and a control sample comprising a mixture of the reference and test samples is labeled with a third label. The signals from the control sample then provide an internal standard that allows more precise determination of the relative concentrations of the proteins of interest in the reference and test samples. The system can thus correct for differences in the labeling efficiencies of the different proteins.

A three-color instrument may be configured with excitation lasers at 488, 532, and 635 nm wavelengths to excite Cy2, Cy3 and Cy5, respectively. The three fluorophores have minimal overlap of their emission spectra, thus minimizing cross-talk in their detection. Accordingly, emission filters having 10 nm FWHM passband widths and peak transmission wavelengths of 522, 570, and 670 nm, respectively, work well with the fluorophores. Various combinations of these and other fluorescent reporters would also work, with varying degrees of crosstalk.

B. Differential RNA Expression Assays

The three or four color assays may also be performed on nucleic acid samples, to provide differential RNA expression (sometimes referred to also as differential gene expression) simultaneously for a reference sample and two or more test samples. This is in contrast to the two-label differential RNA expression assays that are routinely performed on a microarray using immobilized single-strand nucleic acids, such as cDNAs or oligonucleotides, with one label assigned to the reference sample and the second label assigned to the test sample. See, e.g., Schena et al., “Quantitative Monitoring of Gene Expression Patterns with a Complementary DNA Microarray”, Science 270, Oct. 20, 1995., p. 467-470; which is incorporated herein by reference.

Using n multiplex particle types and three or more labels, the current system can simultaneously perform three or more color RNA expression assays on three or more samples. One sample is the reference, and multiple additional samples are test samples. In the example, the reference sample may be normal, while the test samples may be diseased and untreated, or diseased and drug treated, and the multiple test samples can be compared simultaneously to the reference sample.

C. SNP Detection

Nucleic acid assays for SNPs that utilize primer extension as the mechanism for distinguishing the terminal base type of target sequences are also particularly well suited to the systems and methods described herein. Such assays may use four colors of reporter molecules to achieve results that, though highly desirable, could not be produced using known prior particle-based multiplex assay systems that employ at most two labels.

As discussed, known prior particle-based multiplex assays for SNPs are confined to one or two colors. Single-color particle SNP assays require a separate particle type, i.e., one with its own particle ID, for each possible SNP outcome and at least two and possibly four particle types for each SNP location. This complicates assay preparation and can even challenge the particle ID capacity of some systems if, for example, panels of 50 or more SNPs are to be assayed simultaneously (e.g. the Luminex xMAP system is limited to 100 ID codes).

Using the current system, a four-color primer extension SNP assay is performed such that the four possible SNP bases (C, A, T, or G) are extended by terminator labels with four different fluorescent reporters. Accordingly, only one nucleic acid capturing particle type is required per SNP location. The assays are thus not particularly limited by a lack of availability of particle IDs, with panels of 50 or more SNPs readily accommodated. Various sets of four fluorophores, such as FAM, TAMRA, ROX, and Cy5 as well as Cy2, Cy3, Texas Red or Alexa 594, and Cy5 may be used as the independently detectable reporters in the four-color n-multiplexed primer extension assays. Other combinations of four fluorescent reporters may be used, as long as they have appropriately non-overlapping spectra.

D. Multiplex Sandwich Assays

The system's use of a number of labels in multiplex assays can also be advantageous for simplifying assay development for antibody-protein-antibody sandwich assays. As discussed above with reference to FIG. 5, sandwich assays utilize multiple antibody binding sites, or epitopes, of proteins to improve the specificity of the assays. When sandwich assays are multiplexed beyond about 10 or so analytes in parallel, however, it becomes increasingly difficult to make or select secondary, or detector, antibodies that do not have significant cross-reactivity or non-specific binding to non-target assay components. In particular, detector antibodies may bind weakly to the immobilized capture antibodies, and provide false indications of the presence of the proteins of interest. As the number of analytes assayed in multiplex further increases, it becomes more and more difficult to find a set of antibodies in which all of the members of the set exhibit sufficiently negligible amounts of cross reactivity to non-complementary assay components. Accordingly, using known prior systems there is a significant limit to the number of sandwich arrays that can be multiplexed.

In these systems, different pools of particle sets must be used to conduct greater numbers of sandwich assays, so that antibody pairings that have rather significant cross-reactivity are eliminated from the respective pools. The assay development operations are thus made more complex.

The current system simplifies multiplexed sandwich particle-based assay development by dividing a large set of capture and detector antibody pairs into a number of smaller subsets, and labeling each subset with a different color label. Each subset is selected such that there is minimal amount of cross-reactivity within the set. The cross-reactivity between subsets can be essentially ignored, however, because of the use of different colors for detection. The assay development effort is thus substantially reduced from, for example, determining a suitable set of 36 pairs of antibodies to determining three sets of 12 pairs.

The multiple subset sandwich assays may also be produced using two (or more) labels, such that different subsets may be used in multiplexed particle-based sandwich immunoassays.

The subsets may be included in assay kits as components of the pooled particle set, which includes the respective detector antibodies and the associated label molecules. An example is an assay that is performed with monoclonal antibodies. In sequential assays, the detector antibodies and associated label molecules are included in the assay kits in separate vials, to be added appropriately along with associated diluents and wash buffers that are also included in the kits.

The subsets may be used with other multiplex systems, such as microarrays, to achieve the same advantage of simplifying the sandwich assay development. Accordingly, different arrays need not be used to separate the cross reacting capture-detector antibody pairs.

Although specific embodiments have been illustrated and described herein, it will be appreciated by those skilled in the art that any arrangement which is calculated to achieve the same purpose maybe substituted for the specific embodiments shown. This application is intended to cover any adaptations or variations of the present invention, such as the use of devices with controlled wavelengths and optical power, such as LEDs, SLDs, broadband sources with excitation filters, and so forth, in place of or in addition to one or more excitation lasers.

What is claimed is:

1. A particle-based multiplex assay reading system including:
A. a plurality of excitation light sources for exciting three or more assay binding labels and a particle identification image or ratio of labels;
B. three or more detector and wavelength discriminator component pairings for measuring the three or more assay binding labels;
C. one or more objective lenses,
wherein the system uses a detector wavelength discriminator component pairing associated with the wavelength of a given assay binding label to detect a signal produced when a given particle is interrogated by a selected excitation light source.

2. The system of claim 1 wherein the light sources are lasers.

3. The system of claim 1 further including a low resolution detector for detecting a particle identification in the form of a projected code.

4. The system of claim 1 wherein a given detector and wavelength discriminator component pairing and an objective lens are included in a detector module and the system includes three or more detector modules.

5. The system of claim 1 wherein the three or more detector wavelength discriminator component pairings include one detector and multiple filters.

6. The system of claim 5 wherein the multiple filters are included in a filter wheel that is selectively rotated to provide the pairings.

7. A method for developing a particle-based multiplexed sandwich immunoassay kit, including the steps of
A. encoding particles with respective particle identification images or labels;
B. selecting for associating with two or more reporter labels two or more subsets of pairs of capture/detector antibodies;
C. associating the two or more subsets with respective particle identification images or labels;
D. selectively coating the particles that are encoded with the images or labels with at least the respective capture antibodies in accordance with the associated subsets of capture and detector pairs;
E. pooling the coated particles; and
F. providing the detector antibodies and labeling molecules for the respective capture/detector pairs.

8. The method of claim 7 wherein the step of selecting for associating includes selecting for a given subset capture/detector antibody pairs in which there is minimal cross-reactivity.

9. The method of claim 8 wherein the step of selecting for association further includes selecting subsets for associating with three or more labels.

10. The method of claim 7 wherein the step of providing includes providing the detector antibodies and labeling molecules for the pairs in respective vials.

11. The method of claim 10 further includes in the step of providing the detector antibodies, providing associated diluents and wash buffers.

12. The method of claim 7 where in the step of providing includes providing the detector antibodies and labeling molecules for the respective pairs to the pooled particles.

13. A method for producing particle-based multiplexed assays:
a. encoding particles with respective particle identification images or labels to produce encoded particles;
b. selectively coating the encoded particles with specific binding pair members to produce particle types;
c. pooling the particle types;
d. aliquoting portions of the pooled particle types to respective assay vessels;
e. adding to the assay vessels respective samples with three or more reporter labels.

14. The method of claim 13 further including interrogating particles removed from respective assay vessels to detect signals corresponding to the particle identification and the three or more labels.

15. The method of claim 11 wherein the step of interrogating includes selectively exciting the three or more labels.

16. The method of claim 12 further including selectively exciting the three or more labels simultaneously.

17. The method of claim 12 further including selectively exciting the three or more labels serially.

18. A method for producing particle-based multiplexed assays:
a. encoding particles with respective particle identification images or labels to produce encoded particles;
b. selectively coating the encoded particles with a first member of a specific binding pair to produce particle types;
c. pooling the particle types;
d. aliquoting portions of the pooled particle types to respective assay vessels;
e. adding to the assay vessels second members of a specific binding pair with three or more labels.

19. A particle based multiplex nucleic acid primer extension assay including:
particles encoded with respective particle identification images or labels to produce particle types, the particle types corresponding to respective single nucleotide polymorphism locations;
three or more reporter labels associated respectively with three or more terminators or nucleotides.

20. A particle based nucleic acid hybridization assay including:
particles encoded with respective particle identification images or labels; and
nucleic acid probes associated with three or more labels.
